(19) United States
(12) Patent Application Publication
(10) Pub. No.: US 2007/0166821 A1
(43) Pub. Date: Jul. 19, 2007

(54) Serum production system

(75) Inventors: Richard A. Paniccia, Fort Collins, CO (US); Brent R. Bearden, Fort Collins, CO (US); Wendell R. Leinweber, Fort Collins, CO (US)

Correspondence Address: CR MILES, P.C.;CRAIG R. MILES, 405 MASON COURT, SUITE 119, FORT COLLINS, CO, 80524, US

(73) Assignee: Serum Solutions, Inc.

(21) Appl. No.: 11/297,547

(22) Filed: Dec. 7, 2005

(51) Int. Cl. C12N 5/06

(52) U.S. Cl. 435/325

(57) ABSTRACT

Bovine serum compositions having controlled bovine serum characteristics and a method of producing such bovine serum compositions having such controlled serum characteristics from the whole blood of an offspring of a female bovine mammal.

### BACKGROUND

Bovine serum compositions having controlled bovine serum characteristics and methods of producing such bovine serum compositions having such controlled serum characteristics from the whole blood of an offspring of a female bovine mammal.

There is a large commercial market for conventional bovine serum to supplement cell culture medium due to it high nutritional content. Bovine serum is an extremely complex mixture of many small and large biomolecules with different, physiologically balanced growth-promoting and growth inhibiting activities and only bovine serum having certain composition characteristics may be suitable for certain cell culture applications or for promoting and sustaining growth of certain vertebrate mammalian and insect cells. However, even though there is a large commercial market for conventional bovine serum and conventional bovine serum has been made available for many years, substantial long standing problems with respect to producing bovine serum having composition characteristics suitable for certain cell culture applications, or with respect to controlling certain bovine serum composition characteristics remain unresolved.

A substantial problem with certain conventional bovine serum compositions can be the failure to promote and sustain growth of vertebrate mammalian or insect cells. The composition of bovine serum generated from the whole blood of a bovine donor, whether a bovine fetus or a live bovine animal, can vary unpredictably from donor to donor. A wide variety of environmental circumstances may be involved such as in-uterine death of the bovine fetus; duration of time elapsed after in uterine death of the bovine fetus; birth of the bovine fetus; duration of time elapsed after birth of the bovine fetus; materials ingested by offspring of bovine mammals after birth including without limitation water, colostrum, nutritive supplements, milk, or the like; level of activity of the bovine mammal after birth such as locomotion, suckling, or treatment; health of the bovine mammal after birth, or the like.

For example, conventional bovine serum designated as Newborn Bovine Serum or Newborn Calf Serum generated from whole blood collected between one and fifteen days after birth can exhibit a wide range of immunoglobulin G (IgG) levels, for example, between 1500 milligrams per deciliter of serum (mg/dL) and 2,000 mg/dL, or even greater amounts of IgG mg/dL. Because IgG can be active against bacteria, fungi, and foreign particles, or make isolation of monoclonal antibodies produced by cells cultured in such conventional bovine serum impossible or impracticable, high levels IgG as above-described, can preclude use of this conventional type of bovine serum to promote and maintain certain types of vertebrate mammalian or insect cells or for use with certain cell culture applications. Conventionally, the practice to avoid high levels of IgG, such as above-described, can be to obtain bovine fetal whole blood from bovine fetuses from which conventional bovine serum designated as Bovine Fetal Serum, Fetal Bovine Serum, or the like, (collectively “FBS”) can be produced which can have lower levels of IgG, typically not exceeding 1000 micrograms per milliliter (μg/mL), although certain lots of FBS may contain a greater amount of IgG such as between. about 1800 μg/mL and 1900 μg/mL, or the like.

Similarly, with respect to conventional bovine serum designated as Newborn Bovine Serum or Newborn Calf Serum can exhibit a wide range of total protein levels, for example between 4.5 grams per deciliter (g/dL) and 6.5 g/dL, or total protein levels which can be even greater than 6.5 g/dL. This level of total protein can preclude the use of conventional Newborn Bovine Serum or Newborn Calf Serum to promote and maintain certain types of vertebrate mammalian or insect cells or for use in certain cell culture applications. For example, hybridomas can be dependent upon the concentration of total protein in the cell culture medium, and it has been shown that the yield of monoclonal antibodies can be increased as the total protein in the cell culture medium is decreased. Again, conventional practice to avoid such levels of total protein as above-described can be to utilize conventional bovine serum designated as FBS which may be produced with total protein in the range of 3.0 g/dL to 5.0 g/dL, although certain lots of FBS may contain even higher amounts of total protein.

Even though the level of certain bovine serum characteristics of FBS (such as total protein and IgG) important with respect to the successful culture of vertebrate mammalian and insect cells may be less than conventional bovine serum designated Newborn Bovine Serum or Newborn Calf Serum, FBS can still exhibit a broad range of total protein and IgG values as above-described, or even broader, which manifests in a correspondingly broad range of conventional compositions designated as FBS, all of which may be sold for use in cell culture or other applications. This broad range in values for certain constituents in conventional bovine serum designated FBS may be due to the lack of control over whole blood physiology of the bovine fetuses. Fetuses utilized for FBS production are typically obtained from slaughtered or deceased animals. Because carcass processing may take priority over the collection of fetuses for the production of conventional bovine serum designated FBS, a wide variation in both the fetus condition and the elapsed time prior to collection of fetal whole blood can occur.

This variation in fetus condition and in elapsed time to collection of fetal whole blood from fetuses, or both, can reduce control over fetal whole blood physiology and likely contributes to the significant variation in the level of certain constituents found in bovine serum designated FBS, such as total protein or IgG. In any event, there appear to be limits in the context of slaughter houses (or other abattoirs approved by the United States Department of Agriculture) which have not been recognized or otherwise prevent production of conventional bovine serum designated FBS having lower levels, narrower ranges, or less deviation in regard to values of certain FBS constituents important in sustaining growth of vertebrate mammalian or insect cell lines or important with regard to certain cell culture applications.

Moreover, because slaughter houses and other abattoirs approved by the United States Department of Agriculture generate a large number of fetuses from which fetal whole blood can be obtained for conventional FBS production there appears to be little or no motivation to research or develop alternative methods of bovine serum production to avoid the use of fetuses or to produce bovine serum compositions which as compared to conventional FBS have similar, equivalent, or lesser amounts of certain constituents or as compared to convention FBS have narrow ranges, greater constancy or consistency, or lesser deviation with respect to certain constituents, such as total protein, total globulin, or IgG.

### SUMMARY OF THE INVENTION

Accordingly, a broad object of the invention can be to provide particular embodiments of bovine serum compositions which as compared to conventional bovine serum has lower levels (whether lower absolute values or lower average values) of certain constituents which affect the growth of vertebrate mammalian or insect cell lines such as endotoxin, total protein, total globulins, IgG, or the like.

Another broad object of the invention can be to provide particular embodiments of a bovine serum composition which as compared to conventional bovine serum compositions provides a narrower range of values for the level of certain constituents which affect the growth of vertebrate mammalian or insect cell lines such as endotoxin, total protein, globulins, IgG, or the like.

Another broad object of the invention can be to provide particular embodiments of a bovine serum composition which as compared to conventional FSB provides either a comparable level(s) or lower level(s) (whether lower absolute values or lower average values) or comparable ranges or a narrower ranges of values for the level of certain constituents which affect the growth of vertebrate mammalian or insect cell lines such as endotoxin, total protein, globulins, IgG, or the like.

Another broad object of the invention can be to provide particular embodiments of a bovine serum composition which compared with conventional FSB provides either a comparable level(s) or lower level(s) (whether lower absolute values or lower average values), or comparable ranges or narrower ranges of values for certain constituents which affect the growth of vertebrate mammalian or insect cell lines such as endotoxin, total protein, globulins, IgG, or the like, without using the whole blood of fetuses.

Another broad object of the invention can be to provide particular embodiments of a bovine serum composition which as compared to conventional bovine serum designated as Newborn Bovine Serum, Newborn Calf Serum, or prepared from the whole blood of calves between one day and fifteen days old, provides lower level(s) (whether lower absolute values or lower average values) or narrower ranges of values for the level of certain constituents which affect the growth of vertebrate mammalian or insect cell lines such as endotoxin, total protein, globulins, IgG, or the like.

Another broad object of the invention can be to provide a method of producing a bovine serum composition which as compared to conventional bovine serums designated FSB, Newborn Bovine Serum, Newborn Calf Serum, prepared from the whole blood of calves between one day and fifteen days old, or the like, provides lower level(s) (whether lower absolute values or lower average values) or a narrower range of values for the level of certain constituents which affect the growth of vertebrate mammalian or insect cell lines such as endotoxin, total protein, globulins, IgG, or the like.

Naturally, further objects of the invention are disclosed throughout other areas of the specification, drawings, and claims.

### A BRIEF DESCRIPTION OF THE DRAWINGS

FIG. 1 is a flow diagram of a particular embodiment of the method of the invention generating a bovine serum composition.

### DETAILED DESCRIPTION OF THE PREFERRED EMBODIMENTS

Bovine serum compositions having controlled bovine serum characteristics and a method of producing such bovine serum compositions having such controlled serum characteristics from the whole blood of an offspring of a female bovine mammal.

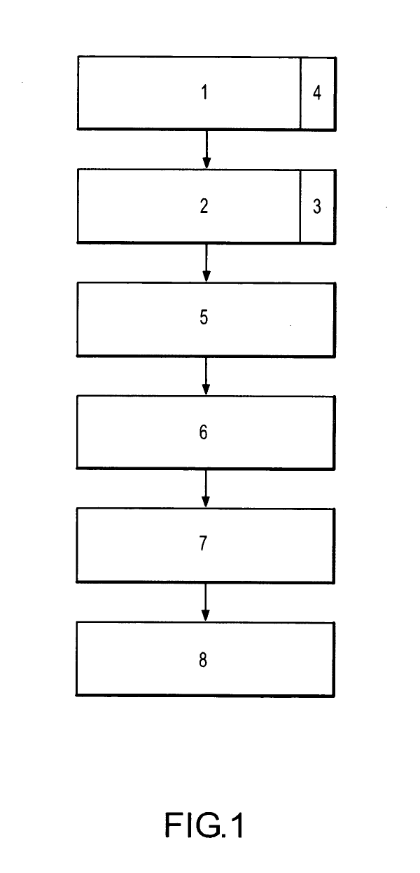

Now referring to FIG. 1, an embodiment of the invention can include a first step (1) of obtaining an offspring of a female bovine mammal. The term “female bovine mammal” is intended to broadly encompass any species of female bovine mammal such as included in the subfamily of bovids (Bovinae) including cattle, oxen, bison, buffalo, and their close relatives and specifically includes female dairy cow breeds such as Ayrshire, Brown Swiss, Guernsey, Holstein, Jersey, and Milking Shorthorn, among others. The term “offspring” is intended to broadly encompass the progeny of the female bovine mammal whether of female or male sex. However, the term offspring specifically does not include fetuses regardless of the source and specifically does not include fetuses obtained from slaughtered female bovine mammals. The term “obtaining” is intended to mean the emergence of the offspring from the birth canal and separation from the female bovine mammal sufficient to perform any or all of the subsequent steps of the invention.

Again referring to FIG. 1, the invention can further include the step of collecting an amount of blood of the offspring of a female bovine mammal (2). Importantly, collecting the amount of blood of the offspring of the female bovine mammal in accordance with the method of the invention can control certain whole blood characteristics which in turn allows production of bovine serum compositions which cannot otherwise be achieved, or cannot be achieved with respect to certain serum composition characteristics (whether total amount, amount per volume, level per volume, average level per volume, amount per volume, or range), or cannot otherwise be achieved with respect to the constancy or consistency of certain serum composition characteristics, or cannot otherwise be achieved without utilizing whole fetal blood obtained from one or a plurality of fetuses. Such serum compositions, such serum composition characteristics, or such constancy or consistency of such serum composition characteristics generated with the whole blood of offspring of female bovine mammals (and without the use fetal whole blood) utilizing the methods encompassed by the invention can be important, or can be critical, with respect promoting or sustaining growth of vertebrate mammalian and insect cells or with respect to certain cell culture applications.

The term “an amount of blood” can broadly encompass any amount of whole blood of the offspring of the female bovine mammal, or all or a portion of the amount of whole blood available from an offspring of a female bovine mammal or a plurality of offspring of a plurality of female bovine mammals, or each of a plurality of discrete amounts of blood maintained separately or a plurality of discrete amounts of blood combined, or combinations or permutations thereof. The term “collecting” is intended to broadly encompass any manner of transferring an amount of blood from the offspring of the female bovine mammal to a receptacle or container. While the invention is not so limited, the receptacle or container can be a blood bag or bleeding bag which can be obtained in a wide variety of constructional forms. For example, the blood bag can be one of or a plurality of the numerous constructional forms available from Anhui Technology Import & Export Co. Ltd, HDO5 Blood Bag, or such as those available from Baldwin Medical, 50 Parkhurst Drive, Knoxfield VIC 3180, Product Code 1053.

As to one non-limiting embodiment of the invention, collecting the amount of blood of the offspring of the female bovine mammal can be accomplished by transferring an amount of removable material, whether solid or liquid, from the surface of the chest area of the offspring of the female bovine mammal. A material removal element, such as a scrapper, can be engaged with the surface of the chest area of the offspring to transfer the amount of removable material. All or a portion of the chest area from which fluid or material was removed can be disinfected by application of a disinfectant such as alcohol. The surface of the chest area from which fluid and material was removed and disinfected should encompass the location at which blood can be transferred from the offspring such as the area about the third and fourth ribs. A needle can be inserted between the third and fourth ribs of the offspring of the female bovine mammal and adjustably located to establish a flow of the amount of blood through the flow path of the needle. The needle can be coupled to a conduit having a conduit flow path fluidicly coupled to the inside of a blood bag. As such, the flow of the amount of blood can be transferred from the offspring of the female bovine mammal through the flow path of the needle and the conduit flow path to be received inside of the blood bag. The front legs or the back legs or both of the offspring of the bovine mammal can be manipulated to generate additional flow of whole blood from the offspring to the blood bag, such as by grasping the front legs and the back legs and generating repeated reciprocal travel of the front legs and the back legs between a first position and a second position.

The flow of the amount of blood can also be made responsive to an amount of vacuum. The amount of vacuum can be applied to all or a part of the external surface of certain constructional forms of the blood bag. In particular applications of the invention, the amount of vacuum can be applied to the external surfaces of the blood bag by locating the blood bag in a vacuum chamber configured to allow the conduit to pass from inside the vacuum chamber to outside the vacuum chamber. A vacuum source, such as a vacuum pump, can be coupled to the vacuum chamber to generate an amount vacuum sufficient to assist in the transfer of whole blood through the flow path of the needle and conduit flow path to be received inside of the blood bag. The amount of vacuum can be adjusted to generate, for example, not greater than 15 mm Hg or can be adjusted to generate between about 10 mm Hg to about 15 mm Hg. During transfer of the amount of blood through the flow path of the needle and the conduit flow path, a first blood bag can be replaced with a second blood bag or replaced by a plurality of blood bags in series to allow the entire amount of blood to be transferred from the offspring of the female bovine mammal. The conduit flow path can be intermittently closed to the flow of the amount of blood during serial blood bag replacement. With respect to conduits having a flexible wall, the flexible wall can be deformed sufficiently with a clamp, forceps, or the like, to establish the flow path of the conduit in the closed condition. Alternately, the flow of blood within a first conduit can be interrupted by generating the closed condition of the conduit and a second needle can be inserted between the third and fourth ribs of the offspring to generate a second flow of blood within a second conduit to be received within the interior of a second blood bag. As to those applications in which vacuum can be applied to the external surface of the blood bag, the amount of vacuum on the first blood bag can be removed and applied to the external surface of the second blood bag. For example, the first blood bag can be removed from the vacuum chamber and the second blood bag located inside the vacuum chamber. After the flow of whole blood from the offspring to the inside of a particular blood bag has been discontinued the conduit flow path of the blood bag can be established in the closed condition. Where the conduit comprises a flexible conduit a knot or a plurality of knots can be established in the conduit to generate the closed condition. The blood bag containing all or a part of the amount of blood can be placed in an ice-water bath. The above-described embodiment of the step of collecting an amount of blood of an offspring of a female bovine mammal may typically take between about ten minutes and about twenty minutes; however, depending upon the expertise of the person performing the step, the offspring from which the amount of blood is collected, or other factors, the step can vary in time duration. Also, while the above-described procedure can be utilized to achieve the step of collecting an amount of blood from the offspring of a female bovine mammal (2), it is not intended that the invention be limited to this particular procedure whether in whole or in part or limited by the order of elements of the procedure as set forth above and alternate methods or procedures which can also achieve the collection of an amount of blood of the offspring of a female bovine mammal inside a receptacle or container can be utilized and are to be considered encompassed by the invention.

As to particular embodiments of the invention the step of collecting an amount of blood of said offspring of said female bovine mammal (2) can further include an amount of time (3) which commences upon obtaining the offspring of the female bovine mammal (1) and after elapse of which the amount of blood of such offspring of such female bovine mammal may not be collected from the offspring mammal. As to these particular embodiments of the invention, the amount of time (3) can include about five minutes, about ten minutes, about fifteen minutes, about twenty minutes, about twenty five minutes, about thirty minutes, about thirty five minutes, about forty minutes, about forty five minutes, about fifty minutes, about fifty five minutes, about sixty minutes, about sixty five minutes, about seventy minutes, about seventy five minutes, about eighty minutes, about eighty five minutes, about ninety minutes, about ninety five minutes, about one hundred minutes, about one hundred and five minutes, about one hundred and ten minutes, about one hundred and fifteen minutes, or about one hundred and twenty minutes.

As other particular embodiments of the invention the step of collecting an amount of blood of said offspring of said female bovine mammal (2) can further include an amount of time (3) which commences upon obtaining the offspring mammal (1) and terminates upon commencement of the step of collecting the amount of blood of the offspring of the female bovine mammal, such as commencement of the above-described procedure for collecting the amount of blood of the offspring of the female mammal or other procedures for collecting the amount of blood of the offspring of the female mammal which take about the same or similar amount of time, but in any event not longer than about one hundred and fifteen minutes. As to these particular embodiments of the invention, the amount of time (3) can include about five minutes, about ten minutes, about fifteen minutes, about twenty minutes, about twenty five minutes, about thirty minutes, about thirty five minutes, about forty minutes, about forty five minutes, about fifty minutes, about fifty five minutes, about sixty minutes, about sixty five minutes, about seventy minutes, about seventy five minutes, about eighty minutes, about eighty five minutes, about ninety minutes, about ninety five minutes, about one hundred minutes, about one hundred and five minutes, about one hundred and ten minutes, about one hundred and fifteen minutes, or about one hundred and twenty minutes.

As to other particular embodiments of the invention the step of collecting an amount of blood of said offspring of said female bovine mammal (2) can further include an amount of time (3) which commences upon obtaining the offspring of the female bovine mammal (1) and terminates upon completion of the step of collecting the amount of blood of the offspring of the female bovine mammal (2). As to these particular embodiments of the invention, the amount of time (3) can include about five minutes, about ten minutes, about fifteen minutes, about twenty minutes, about twenty five minutes, about thirty minutes, about thirty five minutes, about forty minutes, about forty five minutes, about fifty minutes, about fifty five minutes, about sixty minutes, about sixty five minutes, about seventy minutes, about seventy five minutes, about eighty minutes, about eighty five minutes, or about ninety minutes, about ninety five minutes, about one hundred minutes, about one hundred and five minutes, about one hundred and ten minutes, about one hundred and fifteen minutes, or about one hundred and twenty minutes.

As to other particular embodiments of the invention, the step of collecting an amount of blood of said offspring of said female bovine mammal (2) can further include an amount of time (3) commencing upon obtaining the offspring of a female bovine mammal (1) during which the step of collecting an amount of blood of said offspring of said female bovine mammal (2) occurs. As to these particular embodiments of the invention, the amount of time can include about five minutes, about ten minutes, about fifteen minutes, about twenty minutes, about twenty five minutes, about thirty minutes, about thirty five minutes, about forty minutes, about forty five minutes, about fifty minutes, about fifty five minutes, about sixty minutes, about sixty five minutes, about seventy minutes, about seventy five minutes, about eighty minutes, about eighty five minutes, or about ninety minutes, about ninety five minutes, about one hundred minutes, about one hundred and five minutes, about one hundred and ten minutes, about one hundred and fifteen minutes, or about one hundred and twenty minutes.

In a particular embodiment of the invention, the step of collecting an amount of blood of an offspring of a female bovine mammal (2) utilizing the above-described protocol commences not after the elapse of an amount of time (3) of not greater than thirty minutes after obtaining the offspring of the female bovine mammal (1). As above-described, the time duration to perform the step of collecting an amount of blood of an offspring of a female bovine mammal can vary from application to application taking between about ten minutes to about sixty minutes.

In other embodiments of the invention, the step of collecting an amount of blood of said offspring of said female bovine mammal (2) can further include an amount of time (3) which commences upon obtaining the offspring of the female bovine mammal (1) and after elapse of which the amount of blood of such offspring of such female bovine mammal may not be collected from the offspring mammal. As to these particular embodiments of the invention, the amount of time (3) can include about sixty minutes, about one hundred and twenty minutes, about one hundred and eighty minutes, about two hundred and forty minutes, about three hundred minutes, about three hundred and sixty minutes, about four hundred and twenty minutes, or about four hundred and eighty minutes.

In this regard, it can be shown that as to representative offspring of female bovine mammals which are not suckled or otherwise provided nutritional supplements, as further described below, whole blood samples can be collected by the above-described procedure or by withdrawing aliquots from the same offspring at about sixty minutes, about one hundred and twenty minutes, about one hundred and eighty minutes, about two hundred and forty minutes, about three hundred minutes, about three hundred and sixty minutes, about four hundred and twenty minutes, or about four hundred and eighty minutes from which bovine serum compositions encompassed by the invention can be produced.

In any event the above-described examples of the invention are not intended to be limiting with respect to the numerous and wide variety embodiments of the invention which can be practiced which provide an amount of time (3) which further limits the step of collecting an amount of blood from an offspring of a female bovine mammal (2). While one particular embodiment of the invention may limit the step of collecting an amount of blood from an offspring of a female bovine mammal (2) by establishing an amount of time (3) commencing from obtaining the offspring of the female bovine mammal (1) after which the step of collecting the amount of blood of the offspring mammal (2) cannot occur, and while another embodiment of the invention may limit the step of collecting an amount of blood from an offspring of a female bovine mammal (2) by establishing an amount of time (3) commencing from obtaining the offspring of the female bovine mammal (1) to commencing the step of collecting an amount of blood from an offspring of a female bovine mammal (2), and while another particular embodiment of the invention may limit the step of collecting an amount of blood from an offspring of a female bovine mammal (2) by establishing an amount of time (3) commencing from obtaining the offspring of the female bovine mammal (1) after which the step of collecting the amount of blood of the offspring of the female mammal (2) terminates, and while another embodiment of the invention may limit the step of collecting an amount of blood from an offspring of a female bovine mammal (2) by establishing an amount of time (3) commencing from obtaining the offspring of the female bovine mammal (1) during which the step of collecting an amount of blood from an offspring of a female bovine mammal (2) occurs, each of these various embodiments of the invention which limit the step of collecting an amount of blood from an offspring of a female bovine mammal (2) by an amount of time (3) along with any similar or equivalent limitation of the step of collecting an amount of blood from an offspring of a female bovine mammal (2) by an amount of time (3) are each encompassed by the invention.

Again referring to FIG. 1, the step of obtaining the offspring mammal can further include the step of obtaining the offspring mammal prior to the time such offspring of the female bovine mammal ingests any material (4). The term “material” is intended to include materials which the offspring mammal would ingest through suckling the female bovine mammal or would be provided to the offspring of the female bovine mammal to ingest including colostrum, feed, water, or the like. In this regard, the offspring of the female bovine mammal can be separated from the female bovine mammal after birth to avoid suckling and no nutritional supplements, feed, colostrum, or water should be afforded the offspring of the female mammal prior to the step of collecting an amount of blood of the offspring of the female mammal (2).

Again referring to FIG. 1, the invention can further include the step of generating a clot of an amount of clottable material in the amount of blood collected from offspring of the female bovine mammal (5). Typically, the amount of blood of the offspring of the female mammal collected clots under controlled conditions. The clot can comprise a mass of coagulated blood which can contain clottable material such as red blood cells, white blood cells, platelets, fibrin along with those other materials which bind, or otherwise associate to or with such cells, fragments of such cells, or can be entrapped by the fibrin.

Additionally, the invention can include the step of differentiating the clot of clottable material in the amount of blood of the offspring of the female bovine mammal from an amount of non-clottable material in the amount of blood of the offspring of the female bovine mammal (6). Typically, the clottable material can be differentiated from the non-clottable material by centrifugation. The non-clottable material typically referred to as raw serum comprises blood plasma which includes water, proteins, protein fragments, amino acids, salts, lipids, and glucose. However, it is not intended that the invention be limited to differentiating the clottable material from the non-clottable material by centrifugation. Rather, the example of utilizing centrifugation is intended to be illustrative of the variety of approaches which can be used to differentiate clottable material from non-clottable material of an amount of blood obtained from an offspring of a female bovine mammal.

The invention can further include the step of separating the clottable material from the non-clottable material (7). Generally, differentiation of the clottable material from the non-clottable material by centrifugation allows the non-clottable material to be decanted from the clottable material; however, the invention is not so limited and further includes the variety of approaches which can be utilized to separate the clottable material from the non-clottable material of an amount of blood obtained from the offspring of a female bovine mammal.

In an additional step of filtering the non-clottable material (8), the non-clottable material can be passed through a graded range of filter materials of descending pore size. The range of filter materials through which the non-clottable material can be passed can have pore sizes typical of guaze and can descend to pore sizes such as 0.1 μm which can retain bacteria. The filtered non-clottable material can be transferred to sterilized bottles (plastic or glass).

In a particular embodiment of the invention, the amount of blood of the offspring of the female bovine mammal collected into blood bags as above-described can be kept in iced water or otherwise cooled to a similar temperature and transported to a facility for processing as generally above-described to generate the bovine serum compositions encompassed by the invention. An example of such a facility which can process the amount of blood of an offspring of a female bovine mammal is Central Biomedia, Inc., 9900 Pflumm, Lenexa, KS. 66215. The processing and analysis procedure established by Central Biomedia, Inc. for CBI Lot No.: B51285, hereby incorporated by reference, can be utilized to process other amounts of blood collected from the offspring of female bovine mammals as above-described to generate the bovine serum compositions encompassed by the invention. However, it is not intended that this particular example of a processing and analysis procedure be limiting with respect to the wide variety of similar or equivalent processing and analysis procedures utilized by Central Biomedia, Inc. and other similar processing facilitates which can be used to generate bovine serum compositions encompassed by the invention. Additionally, it is not intended that this specific example of a processing and analysis procedure limit the bovine serum compositions encompassed by the invention to the same or similar bovine serum composition characteristics of the particular lot above-described. Rather, bovine serum compositions having bovine serum composition characteristics generated by use of the methods encompassed by the invention are further described below.

Now generally referring to Tables 1-5, the values of particular bovine serum composition characteristics obtained by use of the invention are summarized. Analysis of the bovine serum compositions resulting in the values summarized by Tables 1-3 and 5 were performed by Colorado Veterinary Diagnostic Laboratories, College of Veterinary Medicine and Biomedical Sciences, Colorado State University, Fort Collins, Colo. Original Certificate of Analysis of Lot# A50224A, Certificate of Analysis of Lot # A50901A, Certificate of Analysis of Lot# A41026A, and Laboratory Results IgG Quantitation MCA, each hereby incorporated by reference. Analysis of the bovine serum compositions resulting in the values summarized in Table 4 were performed by Central Biomedia, Inc., 9900 Pflumm, Unit 63, Lenexa, Kanas. Original Certificate of Analysis of Lot# A51103A/MCA hereby incorporated by reference herein.

Now specifically referring to Tables 1-4, the values for total protein with respect to certain lots of the bovine serum compositions generated in accordance with embodiments of the method of invention are shown. Importantly, the stability of specific production rates for certain cell lines (hybridomas, as but one example) can be dependent upon the level of total protein in the cell culture medium contributed by the addition of the bovine serum. Also, with respect to certain cell lines, the yield of monoclonal antibodies can be increased as the total protein in the cell culture medium contributed by the addition of bovine serum decreases. As such, hybridoma cell lines or cell lines used to generate monoclonal antibodies are often cultured in conventional FBS produced from the whole blood of fetuses which can have total protein in the range of about 3.0 g/dL to about 5.0 g/dL.

As can be understood from Tables 1-4, bovine serum compositions generated in accordance with the embodiments of the method of the invention above-described or as set forth by the claims, hereby incorporated by reference, can have total protein limited to an amount not greater than 3.7 g/dL, not greater than 4.2 g/dL, not greater than 4.4 g/dL, or not greater than 4.5 g/dL. Other bovine serum compositions which within a plurality of lots of serum compositions or by combination of a plurality of lots of serum compositions generated utilizing embodiments of the method of the invention can have total protein g/dL limited within a range of 3.7 g/dL to 4.2 g/dL, or limited within a range of between 3.7 g/dL to 4.4 g/dL, or limited within a range of between 3.7 g/dL to 4.5 g/dL or can have total protein g/dL limited within a range of about 3.7 g/dL to about 4.2 g/dL, or limited within a range of between about 3.7 g/dL to about 4.4 g/dL, or limited within a range of between about 3.7 g/dL to about 4.5 g/dL.

The bovine serum compositions generated by embodiments of the method of the invention can have total protein values g/dL that are lower than, similar to, or equivalent to conventional fetal bovine serum, even though fetuses or fetal blood are not utilized to generate the bovine serum compositions encompassed by the invention. As such, the bovine serum compositions generated utilizing embodiments of the method of the invention can be used in cell culture applications which heretofore only conventional FBS has been utilized.

Again specifically referring to Tables 1-3, the values for total globulin with respect to certain lots of the bovine serum compositions generated in accordance with embodiments of the method of invention are shown. Total serum globulin comprises alpha globulins, beta globulins, and gamma globulins. Importantly, because globulins can also interfere with the culture of certain types of cells and the production and isolation of antibodies, levels of globulins g/dL must be limited.

As can be understood from Tables 1-3, serum compositions generated in accordance with the various embodiments of the method of the invention above-described or set forth by the claims, hereby incorporated by reference, can have total globulin g/dL in an amount not greater than 1.5 g/dL, not greater than 1.6 g/dL, not greater than 1.95 g/dL, or not greater than 2.0 g/dL. Other bovine serum compositions which within a plurality of lots of serum compositions or by combination of a plurality of lots of serum compositions generated utilizing the various embodiments of the method of the invention can have total globulin g/dL limited within a range of 1.5 g/dL to 1.6 g/dL, or limited within a range of between 1.5 g/dL to 1.95 g/dL, or limited within a range of between 1.5 g/dL to 2.0 g/dL or can have total globulin g/dL limited within a range of about 1.5 g/dL to about 1.6 g/dL, or limited within a range of between about 1.5 g/dL to about 1.95 g/dL, or limited within a range of between about 1.5 g/dL to about 2.0 g/dL.

The bovine serum compositions generated by embodiments of the method of the invention can have total globulin values g/dL that are lower than, similar to, or equivalent to conventional fetal bovine serum, even though fetuses or fetal blood are not utilized to generate the bovine serum compositions encompassed by the invention.

Now specifically referring to Table 5, the value for IgG with respect to certain lots of the bovine serum compositions generated in accordance with embodiments of the method of the invention are shown. Importantly, IgG can interfere with cell culture and the isolation of monoclonal antibodies. Unfortunately, removal of IgG by artificial methods can further remove or interfere with cell growth promotion components of the bovine serum composition. As such, conventional FBS produced from the whole blood of fetuses is typically used to avoid undesired levels of IgG as above-described.

As can be understood from Tables 5, bovine serum compositions generated in accordance with embodiments of the method of the invention above-described or as set forth by the claims, hereby incorporated by reference, can have IgG limited to an amount not greater than 67 mg/dL or not greater than 100 mg/dL. Other embodiments of the invention can provide serum compositions which within a plurality of lots of serum compositions or by combination of a plurality of lots of serum compositions which can have IgG limited within a range of 50 mg/dL to 100 mg/dL, or limited within a range of about 50 mg/dL to about 100 mg/dL, or limited within a range of about 67 mg/dL to about 100 mg/dL.

As can be understood, the bovine serum compositions generated by embodiments of the method of the invention can have IgG mg/dL that are lower than, similar to, or equivalent to conventional FBS, even though fetuses or fetal blood are not utilized to generate the serum compositions encompassed by the invention.

TABLE 1
Results of Analysis of Lot # A50224A.

| Endotoxin | <0.03 | ng/mL |
|---|---|---|
| Hemoglobin | 14.8 | mg/mL |
| Mycoplasma | Not Detected | |
| pH | 7.5 | |
| Osmolarity | 306 | mOsm/kg |
| Total Protein | 4.2 | g/dL |
| Electrophoretic ID | Characteristic | |
| Viral Characteristics: 9 CFR | | |
| BVD | Not Detected | |
| IBR | Not Detected | |
| PI3 | Not Detected | |
| REO | Not Detected | |
| Parvo | Not Detected | |
| Rabies | Not Detected | |
| BRSV | Not Detected | |
| Blue Tongue | Not Detected | |
| Adeno 1 | Not Detected | |
| Adeno 2 | Not Detected | |
| Bilirubin | 0.1 | mg/dL |
| Creatinine | 2.2 | mg/dL |
| Calcium | 12.9 | mg/dL |
| Glucose | 158 | mg/dL |
| Phosphorous | 7.6 | mg/dL |
| ALP | 178 | IU/L |
| ALT | 10 | IU/L |
| AST | 70 | IU/L |
| Sodium | 141 | meq/L |
| Albumin | 2.7 | g/dL |
| Globulin | 1.6 | g/dL |
| Chloride | 97 | meq/L |
| Triglyceride | 5 | mg/dL |
| Cholesterol | 26 | mg/dL |
| Bicarbonate | 19.4 | meq/L |
| LDH | 747 | IU/L |
| BUN | 13 | mg/dL |
| Uric Acid | 3 | mg/dL |
| Iron | 114 | ug/dL |

TABLE 2
Results of Analysis of Lot # A50901A.

| Endotoxin | <0.03 | ng/mL |
|---|---|---|
| Hemoglobin | 14.6 | mg/mL |
| Mycoplasma | Not Detected | |
| pH | 7.6 | |
| Osmolarity | 305 | mOsm/kg |
| Total Protein | 3.7 | g/dL |
| Electrophoretic ID | Characteristic | |
| Viral Characteristics: 9 CFR | | |
| BVD | Not Detected | |
| IBR | Not Detected | |
| PI3 | Not Detected | |
| REO | Not Detected | |
| Parvo | Not Detected | |
| Rabies | Not Detected | |
| BRSV | Not Detected | |
| Blue Tongue | Not Detected | |
| Adeno 1 | Not Detected | |
| Adeno 2 | Not Detected | |
| Bilirubin | <0.1 | mg/dL |
| Creatinine | 2.3 | mg/dL |
| Calcium | 12.6 | mg/dL |
| Glucose | 135 | mg/dL |
| Phosphorous | 7.4 | mg/dL |
| CK | 239 | IU/L |
| ALP | 178 | IU/L |
| ALT | 10 | IU/L |
| AST | 36 | IU/L |
| GGT | 35 | IU/L |
| SDH | 20 | IU/L |
| Sodium | 147 | meq/L |
| Albumin | 2.5 | g/dL |
| Globulin | 1.5 | g/dL |
| Chloride | 100 | meq/L |
| Triglyceride | 5 | mg/dL |
| Cholesterol | 26 | mg/dL |
| Bicarbonate | 16.1 | meq/L |
| LDH | 747 | IU/L |
| BUN | 13 | mg/dL |
| Uric Acid | 3 | mg/dL |
| Iron | 114 | ug/dL |
| Potassium | 8.6 | meq/L |

TABLE 3
Results of Analysis of Lot # A41026A.

| Endotoxin | 0.048 | ng/mL |
|---|---|---|
| Hemoglobin | 14.9 | mg/mL |
| Mycoplasma | Not Detected | |
| pH | 7.2 | |
| Osmolarity | 302 | mOsm/kg |
| Total Protein | 4.2 | g/dL |
| Electrophoretic ID | Characteristic | |
| Viral Characteristics: 9 CFR | | |
| BVD | Not Detected | |
| IBR | Not Detected | |
| PI3 | Not Detected | |
| REO | Not Detected | |
| Parvo | Not Detected | |
| Rabies | Not Detected | |
| BRSV | Not Detected | |
| Blue Tongue | Not Detected | |
| Adeno 1 | Not Detected | |
| Adeno 2 | Not Detected | |
| Bilirubin | 0.3 | mg/dL |
| Creatinine | 2.6 | mg/dL |
| Calcium | 14.5 | mg/dL |
| Glucose | 112 | mg/dL |
| Phosphorous | 87.4 | mg/dL |
| CK | 372 | IU/L |
| ALP | 178 | IU/L |
| ALT | 10 | IU/L |
| AST | 90 | IU/L |
| GGT | 8 | IU/L |
| SDH | 20 | IU/L |
| Sodium | 164 | meq/L |
| Albumin | 3.0 | g/dL |
| Globulin | 1.95 | g/dL |
| Chloride | 114 | meq/L |
| Triglyceride | 5 | mg/dL |
| Cholesterol | 26 | mg/dL |
| Bicarbonate | 16.1 | meq/L |
| LDH | 747 | IU/L |
| BUN | 13 | mg/dL |
| Uric Acid | 3 | mg/dL |
| Iron | 114 | ug/dL |
| Potassium | 8.7 | meq/L |
| Magnesium | 3.1 | mg/dL |

TABLE 4
Results of Analysis of Lot # A51103A/MCA CBI Lot B51285.

| | |
|---|---|
| Appearance | Dark Amber Colored |
| (Visual) | No Particulate Matter |
| pH (at R.T.) | 7.5 |
| Total Protein (Biuret) | 4.2 g/dL |
| Hemoglobin | 16.8 mg/dL |
| (Three-wavelength Polychromic Analysis) | |
| Endotoxin | 4.8 EU/mL |
| (Gel-clot Formation) | |
| Osmolality | 307 mOsm/kg |
| (Freezing-point Depression) | |
| Sterility | Negative |
| (USP, Membrane Filtration) | |

TABLE 5
Results of Analysis of Lot # A51103A/MCA CBI Lot B51285

| | |
|---|---|
| Serum IgG Quantitation | 67 mg/dL |

As can be easily understood from the foregoing, the basic concepts of the present invention may be embodied in a variety of ways. The invention involves numerous and varied embodiments of a method to produce bovine serum compositions and bovine serum compositions produced by the methods.

As such, the particular embodiments or elements of the invention disclosed by the description or shown in the figures accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “clot” should be understood to encompass disclosure of the act of “clotting”—whether explicitly discussed or not—and, conversely, were there effectively disclosure of the act of “clotting”, such a disclosure should be understood to encompass disclosure of a “clot” and even a “means for clotting.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

Thus, the applicant(s) should be understood to claim at least: i) the serum herein disclosed and described, ii) the related methods of producing the serum disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The claims set forth in this specification are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

The claims set forth below are intended describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

We claim:

1. A method of generating an amount of serum, comprising the steps of:
a. obtaining an offspring of a female bovine mammal;
b. collecting an amount of blood of said offspring of said female bovine mammal;
c. generating a clot of an amount of clottable material in said amount of blood;
d. differentiating said clot of said amount of clottable material in said amount of blood from an amount of non-clottable material in said amount of blood; and
e. separating said clot of said amount of clottable material in said amount of blood from said amount of non-clottable material in said amount of blood to generate said amount of serum.

2. A method of generating an amount of serum as described in claim 1, wherein said step of collecting an amount of blood of said offspring of said female bovine mammal comprises the step of collecting an amount of blood of said offspring of said female bovine mammal not after the elapse of an amount of time after obtaining said offspring of said female bovine mammal selected from the group consisting of about five minutes, about ten minutes, about fifteen minutes, about twenty minutes, about twenty five minutes, about thirty minutes, about thirty five minutes, about forty minutes, about forty five minutes, about fifty minutes, about fifty five minutes, about sixty minutes, about sixty five minutes, about seventy minutes, about seventy five minutes, about eighty minutes, about eighty five minutes, or about ninety minutes, about ninety five minutes, about one hundred minutes, about one hundred and five minutes, about one hundred and ten minutes, about one hundred and fifteen minutes, or about one hundred and twenty minutes.

3. A method of generating an amount of serum as described in claim 2, wherein said step of collecting an amount of blood of said offspring of said female bovine mammal further comprises the step of initiating collection of an amount of blood of said offspring of said female bovine mammal not after elapse of an amount of time after obtaining said offspring of said female bovine mammal selected from the group consisting of about five minutes, about ten minutes, about fifteen minutes, about twenty minutes, about twenty five minutes, about thirty minutes, about thirty five minutes, about forty minutes, about forty five minutes, about fifty minutes, about fifty five minutes, about sixty minutes, about sixty five minutes, about seventy minutes, about seventy five minutes, about eighty minutes, about eighty five minutes, or about ninety minutes, about ninety five minutes, about one hundred minutes, about one hundred and five minutes, about one hundred and ten minutes, about one hundred and fifteen minutes, or about one hundred and twenty minutes.

4. A method of generating an amount of serum as described in claim 3, wherein said step of collecting an amount of blood of said offspring of said female bovine mammal further comprises the step of terminating collection of an amount of blood of said offspring of said female bovine mammal not after elapse of an amount of time after obtaining said offspring of said female bovine mammal selected from the group consisting of about five minutes, about ten minutes, about fifteen minutes, about twenty minutes, about twenty five minutes, about thirty minutes, about thirty five minutes, about forty minutes, about forty five minutes, about fifty minutes, about fifty five minutes, about sixty minutes, about sixty five minutes, about seventy minutes, about seventy five minutes, about eighty minutes, about eighty five minutes, or about ninety minutes, about ninety five minutes, about one hundred minutes, about one hundred and five minutes, about one hundred and ten minutes, about one hundred and fifteen minutes, or about one hundred and twenty minutes.

5. A method of generating an amount of serum as described in claim 1, wherein said step of collecting an amount of blood of said offspring of said female bovine mammal occurs within a duration of time after obtaining said offspring of said female bovine mammal selected from the group consisting of a duration of time of about five minutes, a duration of time of about ten minutes, a duration of time of about fifteen minutes, a duration of time of about twenty minutes, a duration of time of about twenty five minutes, a duration of time of about thirty minutes, a duration of time of about thirty five minutes, a duration of time of about forty minutes, a duration of time of about forty five minutes, a duration of time of about fifty minutes, a duration of time of about fifty five minutes, a duration of time of about sixty minutes, about sixty five minutes, about seventy minutes, about seventy five minutes, about eighty minutes, about eighty five minutes, or about ninety minutes, about ninety five minutes, about one hundred minutes, about one hundred and five minutes, about one hundred and ten minutes, about one hundred and fifteen minutes, or about one hundred and twenty minutes.

6. A method of generating an amount of serum as described in claim 1, wherein said step of collecting an amount of blood of said offspring of said female bovine mammal further comprises the step of terminating collection of an amount of blood of said offspring of said female bovine mammal not after elapse of an amount of time after obtaining said offspring of said female bovine mammal selected from the group consisting of about sixty minutes, about one hundred and twenty minutes, about one hundred and eighty minutes, about two hundred and forty minutes, about three hundred minutes, about three hundred and sixty minutes, about four hundred and twenty minutes, and about four hundred and eighty minutes.

7. A method of generating an amount of serum as described in claim 1, wherein said step of collecting an amount of blood of said offspring of said female bovine mammal occurs prior to generation of total protein in said amount of serum not greater than 4.5 g/dL.

8. A method of generating an amount of serum as described in claim 6, wherein said step of collecting an amount of blood of said offspring of said female bovine mammal occurs prior to generation of an amount of globulins in said amount of serum not greater than about 2.0 g/dL.

9. A method of generating an amount of serum as described in claim 7, wherein said step of collecting an amount of blood of said offspring of said female bovine mammal occurs prior to generation of an amount of gamma globulin in said amount of serum not greater than about 100 mg/dL

10. A method of generating an amount of serum as described in claim 4, 5, or 6, wherein said step of obtaining an offspring of a female bovine mammal comprises the step of obtaining said offspring of said female bovine mammal prior to said female bovine mammal suckling said offspring.

11. A method of generating an amount of serum as described in claim 10, further comprising the step of obtaining said offspring of said female bovine mammal prior to said offspring of said female bovine mammal ingesting colostrum.

12. A method of generating an amount of serum as described in claim 12, wherein said offspring of said female bovine mammal comprises an offspring of a female bovine dairy mammal.

13 A method of generating an amount of serum as described in claim 13, wherein said offspring of said female bovine dairy mammal comprises a male offspring of a bovine dairy mammal.

13. A method of generating an amount of serum, comprising the steps of:
a. obtaining an offspring of a female bovine mammal;
b. collecting an amount of blood of said offspring of said female bovine mammal, wherein collection of said amount of blood of said offspring of said female bovine mammal occurs within a duration of time between birth and about thirty minutes of birth;
c. generating a clot of an amount of clottable material in said amount of blood;
d. differentiating said clot of said amount of clottable material in said amount of blood from an amount of non-clottable material in said amount of blood; and
e. separating said clot of said amount of clottable material in said amount of blood from said amount of non-clottable material in said amount of blood to generate said amount of serum.

14. A bovine serum composition produced in accordance with claim 1.
